(19) United States
(12) Patent Application Publication
(10) Pub. No.: US 2026/0284297 A1
(43) Pub. Date: Sep. 24, 2026

(54) SYSTEMS AND METHODS FOR USE IN DELIVERY OF FLUID TO A PATIENT USING TUBING TO HOLD CONTRAST MEDIA PRIOR TO SALINE FLUSH

(71) Applicant: Bayer Healthcare LLC, Whippany, NJ (US)

(72) Inventors: KEVIN COWAN, ALLISON PARK, PA (US); ARTHUR UBER, III, PITTSBURGH, PA (US); DAVID GRIFFITHS, PITTSBURGH, PA (US)

(21) Appl. No.: 19/471,917

(22) PCT Filed: Apr. 3, 2024
(86) PCT No.: PCT/US2024/022782

§ 371 Date: Oct. 2, 2025

Related U.S. Application Data
(60) Provisional application No. 63/457,940, filed on Apr. 7, 2023.

(51) Int. Cl. A61M 5/00; A61M 5/14; A61M 5/31

(52) U.S. Cl. CPC A61M 5/007; A61M 5/1413; A61M 5/3146; A61M 2005/1403

(57) ABSTRACT

A system for use in delivery of precise, small volumes of medical fluid to a patient is described. The system includes a first tubing set having a first length of tubing and a second length of tubing, a first connector member positioned at the proximal end of the first length of tubing configured to connect to a syringe, and a valve assembly positioned at the distal end of the first length of tubing. The system utilizes a section of the first length of tubing to measure and contain the small volumes of medical fluid for flushing into a patient through the valve assembly and second length of tubing.

### CROSS-REFERENCE TO RELATED APPLICATION

This application claims the benefit of U.S. Provisional Patent Application No. 63/457,940, filed 7 Apr. 2023, the disclosure of which is incorporated by reference in its entirety.

### BACKGROUND

### Field of the Disclosure

The present disclosure relates to devices, systems, and methods associated with medical devices for use in delivery of a medical fluid to a patient, and in particular, devices, systems, and methods for measuring precise and/or small volumes, holding precise and/or small volumes and/or delivering precise and/or small volumes of medical fluid using fluid injection systems.

### Description of Related Art

An injection system, such as a fluid injection system (e.g., a medical fluid delivery system) may be used by a medical practitioner in a medical diagnostic procedure and/or a medical therapeutic procedure. For example, the medical practitioner may use the fluid injection system to inject a patient with one or more medical fluids. The fluid injection system may be used for pressurized injection of a medical fluid, such as a radiological contrast material (e.g., a contrast agent, contrast media, etc.), and/or a flushing agent, such as saline, in medical imaging procedures, such as angiography (CV), computed tomography (CT), ultrasound, magnetic resonance imaging (MRI), and positron emission tomography (PET). A fluid injection system may also be used to deliver medical fluids or liquid medicaments to a patient during other procedures and/or treatments, including nuclear medicine imaging, molecular imaging, radiopharmaceutical injection, and cardiovascular localized therapy. In some instances, the fluid injection system is designed to deliver an amount of a medical fluid at one or more flow rates into a patient.

In a medical treatment or procedure, the amount of medical fluid that must be delivered to a patient is a dose measured by volume. In some instances, the precise measurement of a dose to be delivered to a patient is critical for proper imaging, or proper treatment, and for avoiding adverse reactions or complications to patient health. Improvements to imaging systems and contrast agents also allow for or require lower doses of contrast media to be used for procedures while still achieving accurate and detailed images. For example, imaging systems are increasingly more sensitive to contrast agents, predictive artificial intelligence algorithms allow for lower volumes of contrast media to be used, and contrast agents are being developed with higher relaxivity. These factors allow for lower volumes of contrast media to be used during procedures. However, known fluid injection systems often include larger injectors and higher volume syringes and do not provide for adequate volume control or accurate measurement of the precise doses indicated for patient delivery. For example, MRI procedures for an average adult male may have previously required more than 30 mL of liquid contrast media for accurate imaging. In light of improvements to imaging systems and contrast agents, an MRI procedure for an average adult male may require less than 30 mL or even less than 20 mL of contrast media. In some instances, a dose for an average adult male may be as little as 8 mL to 15 mL. In some instances, a dose for a pediatric patient may be as little as 1 mL to 2 mL of contrast media. In some instances, certain procedures or treatments may require precise doses of medical fluids as little as 0.1 mL to 0.5 mL.

In addition, use of excessive volumes of contrast media is a non-trivial expense to care providers and, ultimately, their patients. Accordingly, volume is a significant contributor to the cost associated with making, distributing, and using contrast media, and limiting volumetric use of contrast media is crucial to limiting costs to healthcare professionals and patients.

In view of the foregoing, there exists a need for devices, systems, and methods for measuring precise and/or small volumes, holding precise and/or small volumes and/or delivering precise and/or small volumes of medical fluid using fluid injection systems.

### SUMMARY

Accordingly, aspects of the present disclosure are directed to non-limiting embodiments of devices, systems, and methods for measuring precise and/or small volumes, holding precise and/or small volumes and/or delivering precise and/or small volumes of medical fluid using fluid injection systems.

According to an aspect of the disclosure, a system for use in delivery of fluid to a patient includes a first tubing set having a first length of tubing and a second length of tubing, the first length of tubing and the second length of tubing each having a proximal end and a distal end. A first connector member is positioned at the proximal end of the first length of tubing, the first connector member configured to connect to a flushing syringe, and a valve assembly is positioned at the distal end of the first length of tubing. The valve assembly includes a proximal port, a distal port, and a third port. A second connector connects the distal end of the first length of tubing to the proximal port. A third connector member is connected to the distal port. A fourth connector member connects a first swabable valve and the second length of tubing to the third port. The system also includes a container containing a medical fluid, which is removably connected to the first swabable valve. The valve assembly selectively provides a fluid connection between the proximal port, the distal port, and the third port.

According to another aspect of the disclosure, a system for use in delivery of fluid to a patient includes a first length of tubing having a proximal end and a distal end, a first syringe connected to the proximal end of the first length of tubing, and a valve assembly positioned at the distal end of the first length of tubing. The valve assembly includes a first port, a second port, and a third port; connector members connected to each of the first port, the second port, and the third port; and a first swabable valve connected to the third port. The second port is connected to a proximal end of a second tubing set and a catheter configured to be fluidly connectable to a patient. The distal end of the first length of tubing is connected to the first port, and a container containing a medical fluid is removably connected to the valve assembly.

According to another aspect of the disclosure, a fluid injection system includes a housing, at least one syringe, at least one drive member, and at least one controller programmed or configured to control actuation of the at least one drive member. The fluid injection system further includes an administration line, a first length of tubing having a proximal end and a distal end, and a first connector member positioned at the proximal end of the first length of tubing, the first connector member configured to connect to the at least one syringe of the fluid injection system. Further, the system includes a valve assembly positioned at the distal end of the first length of tubing. The valve assembly includes a proximal port, a distal port, and a third port, a second connector member connecting the distal end of the first length of tubing to the proximal port, a third connector member connecting the distal port to a proximal end of the administration line, and a fourth connector member connecting a first swabable valve to the third port. The first swabable valve removably connects a container containing a medical fluid to the third port, and the valve assembly selectively provides a fluid connection between the proximal port, the distal port, and the third port. The medical fluid is a liquid contrast media and the at least one syringe is filled with a flushing fluid.

According to another aspect of the disclosure, a method of using a fluid injection system includes providing a fluid injection system. The method further includes priming at least one syringe of the system by pushing a flushing fluid into the at least one syringe, connecting an administration line to a third connector member and to a proximal port of a valve assembly, and connecting the proximal end of a first length of tubing to the at least one syringe. Further, the method includes priming the fluid injection system by actuating at least one drive member and pushing a flushing fluid contained in the at least one syringe through the first length of tubing and through the administration line. The method further includes connecting a container containing a medical fluid to a first swabable valve, actuating at least one drive member to draw a volume of the medical fluid into the first length of tubing, and actuating the at least one drive member to push the volume of medical fluid from the first length of tubing through the administration line and to the patient.

Non-limiting illustrative examples of embodiments of the present disclosure will now be described in the following numbered clauses.

Clause 1: A system for use in delivery of fluid to a patient, the system comprising: a first tubing set having a first length of tubing and a second length of tubing, the first length of tubing and the second length of tubing each having a proximal end and a distal end; a first connector member positioned at the proximal end of the first length of tubing, the first connector member configured to connect to a flushing syringe; and a valve assembly positioned at the distal end of the first length of tubing, the valve assembly comprising: a proximal port, a distal port, and a third port; a second connector member connecting the distal end of the first length of tubing to the proximal port; a third connector member connected to the distal port; and a fourth connector member connecting a first swabable valve and the second length of tubing to the third port, wherein a container containing a medical fluid is removably connected to the first swabable valve, and wherein the valve assembly selectively provides a fluid connection between the proximal port, the distal port, and the third port.

Clause 2: The system of clause 1, wherein the third connector member connected to the distal port is connected to a proximal end of a second tubing set, wherein the second tubing set further comprises a one-way check valve configured to allow fluid flow toward the distal end of the second tubing set.

Clause 3: The system of clause 1 or 2, wherein the second tubing set is an administration line connected to a catheter configured to be fluidly connectable to a patient.

Clause 4: the system of any of clauses 1-3, wherein the administration line

is a single-use set.

Clause 5: The system of any of clauses 1-4, wherein the first tubing set and the valve assembly is a multi-use set such that it maintains sterility and is suitable for use with more than one patient.

Clause 6: The system of any of clauses 1-5, wherein the first connector member is connected to the flushing syringe and the flushing syringe is a manual syringe, an auto-injector, or a syringe connected to a fluid injection system.

Clause 7: The system of any of clauses 1-6, wherein the flushing syringe contains a flushing fluid, and wherein the valve assembly provides a fluid connection between the proximal port of the first tubing set and distal port of the second tubing set, and actuation of a plunger or a piston of the flushing syringe pushes the flushing fluid through the second tubing set to flush the administration line.

Clause 8: The system of any of clauses 1-7, wherein at least one of the first connector member, the second connector member, the third connector member, and the fourth connector member comprise at least one of a one way check valve and a luer connector.

Clause 9: The system of any of clauses 1-8, wherein the first length of tubing is sized to hold a volume of up to about 30 mL of the medical fluid.

Clause 10: The system of any of clauses 1-9, wherein the first length of tubing is sized to a hold a volume ranging from 0.1 mL to about 15 ml of the medical fluid.

Clause 11: The system of any of clauses 1-10, wherein the first length of tubing is sized to a hold a volume ranging from 0.1 mL to about 5 mL of the medical fluid.

Clause 12: The system of any of clauses 1-11, wherein the first length of tubing is comprised of a translucent polymeric material such that the medical fluid in the first length of tubing is visible to a user and/or a sensor of a fluid injection system.

Clause 13: The system of any of clauses 1-12, wherein the first length of tubing further comprises indicia configured to provide a volumetric measurement of the amount of medical fluid in the first length of tubing.

Clause 14: The system of any of clauses 1-13, further comprising a volume control element positioned between the container containing the medical fluid and the first length of tubing, and wherein the valve assembly provides a fluid connection between the third port and the proximal port, and the volume control element selectively controls fluid flow between the container and the first length of tubing such that a measured volume of the medical fluid flows from the container into the first length of tubing.

Clause 15: The system of any of clauses 1-14, wherein the container containing the medical fluid is a second syringe, and wherein the valve assembly provides a fluid connection between the third port and the proximal port, and the second syringe selectively controls fluid flow between the second syringe and the first length of tubing such that a measured volume of the medical fluid flows from the second syringe to the first length of tubing.

Clause 16: The system of any of clauses 1-15, wherein a spike is connected to the swabable valve of the third port, wherein the container containing the medical fluid is pouch, a bag, a bottle, or another vessel configured to receive the spike, and wherein when the container receives the spike it is fluidly connected to the valve assembly.

Clause 17: The system of any of clauses 1-16, wherein the first connector member is connected to the flushing syringe connected to a fluid injection system, and the fluid injection system is programmed or controlled to actuate a piston of the flushing syringe to draw a predetermined volume of the medical fluid from the container into the first length of tubing.

Clause 18: The system of any of clauses 1-17, wherein the second syringe comprises a pulsing syringe configured to selectively control fluid flow from the second syringe to the first length of tubing by delivering a predetermined volume of the medical fluid a predetermined number of times from the pulsing syringe into the first length of tubing.

Clause 19: The system of any of clauses 1-18, wherein the pulsing syringe is connected to the fluid injection system and the fluid injection system is programmed or controlled to actuate a piston of the pulsing syringe to deliver the predetermined volume of the medical fluid the predetermined number of times from the pulsing syringe to the first length of tubing.

Clause 20: The system of any of clauses 1-19, wherein the predetermined volume of the medical fluid ranges from 0.1 mL to about 1 mL.

Clause 21: The system of any of clauses 1-20 for use in delivery of fluid to a patient, the system comprising: a first length of tubing having a proximal end and a distal end; a first syringe connected to the proximal end of the first length of tubing; a valve assembly positioned at the distal end of the first length of tubing, the valve assembly comprising: a first port, a second port, and a third port; connector members connected to each of the first port, the second port, and the third port; and a first swabable valve connected to the third port; wherein the second port is connected to a proximal end of a second tubing set and a catheter configured to be fluidly connectable to a patient, wherein the distal end of the first length of tubing is connected to the first port, wherein a container containing a medical fluid is removably connected to the valve assembly.

Clause 22: The system of any of clauses 1-21, wherein the system further comprises: a second syringe; and an additional connector member between the third port and the second syringe, wherein the second syringe is removably connected to the additional connector member and the third port, and wherein the connector members and the additional connector member are configured to selectively control fluid flow between the container, the second syringe, and the first length of tubing, such that a measured volume of the medical fluid is allowed to flow from the container to the first length of tubing, or from the second syringe to the first length of tubing.

Clause 23: The system of any of clauses 1-22, wherein the container is disconnected from the swabable valve and the swabable valve receives a valve cap, or wherein the second syringe is disconnected from additional connector member and the additional connector member receives a connector member cap.

Clause 24: The system of any of clauses 1-23, wherein the system further comprises a third length of tubing having a proximal end and a distal end, and the container comprises a pre-filled component positioned between the first length of tubing and the valve assembly, wherein the proximal end of the third length of tubing is connected to the first syringe, and the distal end of the third length of tubing is connected to the second tubing set, wherein the third length of tubing is a bypass line configured to deliver a flushing fluid to the second tubing set and to the patient, and wherein the flushing fluid is delivered through the first length of tubing and the pre-filled component is actuated to deliver the medical fluid through the first length of tubing to the second tubing set and to the patient.

Clause 25: The system of any of clauses 1-24, wherein the first length of tubing further comprises a syringe having a two-sided plunger.

Clause 26: The system of any of clauses 1-25, wherein the first length of tubing further comprises a rolling diaphragm, Clause 27: The system of any of clauses 1-26 implemented in a fluid injection system, the fluid injection system comprising: a housing, at least one syringe, at least one drive member, and at least one controller programmed or configured to actuate the at least one drive member; an administration line; a first length of tubing having a proximal end and a distal end; a first connector member positioned at the proximal end of the first length of tubing, the first connector member configured to connect to the at least one syringe of the fluid injection system and a valve assembly positioned at the distal end of the first length of tubing, the valve assembly comprising: a proximal port, a distal port, and a third port; a second connector member connecting the distal end of the first length of tubing to the proximal port; a third connector member connecting the distal port to a proximal end of the administration line; and a fourth connector member connecting a first swabable valve to the third port, the first swabable valve removably connecting a container containing a medical fluid to the third port, wherein the valve assembly selectively provides a fluid connection between the proximal port, the distal port, and the third port, and wherein the medical fluid is a liquid contrast media and the at least one syringe is filled with a flushing fluid.

Clause 28: The fluid injection system of any of clauses 1-27, wherein the valve assembly provides a fluid connection between the proximal port and distal port, and actuation of the at least one drive member pushes the flushing fluid from the at least one syringe through the administration line to flush the administration line.

Clause 29: The fluid injection system of any of clauses 1-28, wherein the valve assembly further provides a fluid connection between the third port and the proximal port such that a volume of the liquid contrast media is drawn into the first length of tubing by actuation of the at least one drive member, or wherein the container is a second syringe and a volume of the liquid contrast media is pushed into the first length of tubing by actuation of a plunger of the second syringe.

Clause 30: The fluid injection system of any of clauses 1-29, wherein the valve assembly further provides a fluid connection between the proximal port and the distal port such that a volume of the liquid contrast media is pushed from the first length of tubing through the administration line and to the patient by actuation of the at least one drive member.

Clause 31: The fluid injection system of any of clauses 1-30, further comprising a measurement sensor configured to measure the volume of liquid contrast media that is drawn into the first length of tubing, and wherein measurement sensor is configured to communicate with the controller when a predetermined volume of liquid contrast media is drawn or pushed into the first length of tubing.

Clause 32: The fluid injection system of any of clauses 1-31, wherein the fluid injection system further comprises air sensors positioned on a second length of tubing and/or the administration line, and wherein the air sensors are configured to detect air in the second length of tubing and/or the administration line, and communicate the detection of air with the controller of the fluid injection system.

Clause 33: A method of using a fluid injection system of any of clauses 1-32, the method comprising: providing a fluid injection system comprising: a housing, at least one syringe, at least one drive member, and at least one controller programmed or configured to actuate the at least one drive member; an administration line; a first length of tubing having a proximal end and a distal end; a first connector member positioned at the proximal end of the first length of tubing, the first connector member configured to connect to the at least one syringe of the fluid injection system; and a valve assembly positioned at the distal end of the first length of tubing, the valve assembly comprising: a proximal port, a distal port, and a third port; a second connector member connecting the distal end of the first length of tubing to the proximal port; a third connector member connected to the distal port and configured to connect to a proximal end of the administration line; and a fourth connector member connecting a first swabable valve to the third port; priming the at least one syringe by pushing a flushing fluid into the at least one syringe; connecting the administration line to the third connector member and the proximal port of the valve assembly; connecting the proximal end of the first length of tubing to the at least one syringe of the fluid injection system; priming the fluid injection system by actuating the at least one drive member and pushing a flushing fluid contained in the at least one syringe through the first length of tubing and through the administration line; connecting a container containing a medical fluid to the first swabable valve; actuating the at least one drive member to draw a volume of the medical fluid into the first length of tubing; and actuating the at least one drive member to push the dose of medical fluid from the first length of tubing through the administration line and to the patient.

Further details and advantages of the various examples described in detail herein will become clear upon reviewing the following detailed description of the various examples in conjunction with the accompanying drawing figures.

### BRIEF DESCRIPTION OF THE DRAWINGS

Additional advantages and details are explained in greater detail below with reference to the exemplary embodiments that are illustrated in the accompanying figures, in which:

FIG. 1 illustrates a non-limiting embodiment of an environment, inclusive of a fluid injection system and a medical imaging system in accordance with an aspect of the present disclosure;

FIG. 2 illustrates a non-limiting embodiment of a fluid injection system in accordance with an aspect of the present disclosure;

FIG. 3 is an enlarged view of an injector head unit of the fluid injection system shown in FIG. 2;

FIG. 4A illustrates an implementation of a non-limiting embodiment of a system for use with a fluid injection system in accordance with an aspect of the present disclosure;

FIGS. 4B-4D illustrate implementations of various components including a syringe, a single-use tubing set, and a multi-use set of a non-limiting embodiment of the system of FIG. 4A in accordance with an aspect of the present disclosure;

FIG. 4E illustrates implementation of a non-limiting embodiment of the system shown in FIGS. 4A-4D during an operation in which a flushing fluid is used to prime the syringe, the single-use tubing set, and the multi-use set in accordance with an aspect of a method of the present disclosure;

FIG. 4F illustrates implementation of a non-limiting embodiment of the system shown in FIGS. 4A-4E during a filling operation in which a volume of contrast media is drawn into a portion of the multi-use set in accordance with an aspect of a method of the present disclosure;

FIG. 4G illustrates implementation of a non-limiting embodiment of the system shown in FIGS. 4A-4F during an injection procedure in which the desired dose of contrast media is flushed from the multi-use set to a destination in accordance with an aspect of a method of the present disclosure;

FIGS. 5-12 are schematic illustrations of non-limiting embodiments of a system for use with a fluid injection system and various components thereof in accordance with aspects of the present disclosure;

FIG. 13 is a sequence diagram of a non-limiting embodiment of a method for using devices, systems, and/or fluid injection systems in accordance with aspects s of the present disclosure.

Corresponding reference characters indicate corresponding parts throughout the several views. The exemplifications set out herein illustrate exemplary embodiments of the disclosure, and such embodiments are not to be construed as limiting the scope of the disclosure in any manner.

### DETAILED DESCRIPTION

It is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects of the disclosure. Hence, specific dimensions and other physical characteristics related to the embodiments or aspects disclosed herein are not to be considered as limiting unless otherwise indicated.

For purposes of the description hereinafter, the terms “end,” “upper,” “lower,” “right,” “left,” “vertical,” “horizontal,” “top,” “bottom,” “lateral,” “longitudinal,” and derivatives thereof shall relate to embodiments or aspects of the disclosure as they are oriented in the drawing figures. However, it is to be understood that embodiments or aspects may assume various alternative variations and step sequences, except where expressly specified to the contrary. Spatial or directional terms, such as “left”, “right”, “inner”, “outer”, “above”, “below”, and the like, are not to be considered as limiting as the disclosure can assume various alternative orientations.

All numbers used in the specification and claims are to be understood as being modified in all instances by the term “about”. The terms “approximately”, “about”, and “substantially” mean a range of plus or minus ten percent of the stated value.

As used herein, the term “at least one of” is synonymous with “one or more of.” For example, the phrase “at least one of A, B, and C” means any one of A, B, and C, or any combination of any two or more of A, B, and C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C. Similarly, as used herein, the term “at least two of” is synonymous with “two or more of.” For example, the phrase “at least two of D, E, and F” means any combination of any two or more of D, E, and F. For example, “at least two of D, E, and F” includes one or more of D and one or more of E; one or more of D and one or more of F; one or more of E and one or more of F; or one or more of all of D, E, and F.

No aspect, component, element, structure, act, step, function, instruction, and/or the like used herein should be construed as critical or essential unless explicitly described as such. Also, as used herein, the articles “a” and “an” are intended to include one or more items and may be used interchangeably with “one or more” and “at least one.” As used in the specification and the claims, the singular form of “a,” “an,” and “the” include plural referents, unless the context clearly dictates otherwise. Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. Further, the phrase “based on” is intended to mean “based at least partially on” unless explicitly stated otherwise.

When used in relation to a component of a fluid injection system such as a fluid reservoir, a syringe, or a fluid line, the term “distal” refers to a portion of said component nearest to a patient. When used in relation to a component of a fluid injection system such as a fluid reservoir, a syringe, or a fluid line, the term “proximal” refers to a portion of said component nearest to the injector of the fluid injection system (i.e., the portion of said component farthest from the patient). When used in relation to a syringe of a multi-patient disposable set, the term “proximal” refers to a portion of a syringe nearest a piston for delivering fluid from a syringe. When used in relation to a component of a fluid injection system such as a fluid reservoir, a syringe, or a fluid line, the term “upstream” refers to a direction away from the patient and towards the injector of the fluid injection system. When used in relation to a component of a fluid injection system such as a fluid reservoir, a syringe, or a fluid line, the term “downstream” refers to a direction towards the patient and away from the injector of the fluid injection system.

Embodiments of the present disclosure are generally directed to systems for use with a fluid injection system, the systems may include a first length of tubing and a second length of tubing, a first connector member positioned at the proximal end of the first length of tubing configured to connect to a syringe, and a valve assembly positioned at the distal end of the first length of tubing. The valve assembly may include a proximal port, a distal port, a third port, a second connector member connecting the first length of tubing to the proximal port, a third connector member connected to the distal port, and a fourth connector member connecting a first swabable valve to the third port. A container containing a medical fluid may be removably connected to the first swabable valve. The valve assembly may selectively provide a fluid connection between the proximal port, the distal port, and the third port. According to various embodiments, the third port of the valve assembly may be a T-port or a Y-port extending perpendicular or at an angle, respectively, to the axis defined between the proximal port and the distal port.

In this way, embodiments of the present disclosure allow for measuring precise and/or small volumes, holding precise and/or small volumes, and/or delivering precise and/or small volumes of medical fluid, such as small volumes of contrast media, for use in fluid injection systems and for injection into a patient.

Referring to the drawings in which like reference characters refer to like parts throughout the several views thereof, the present disclosure is generally directed to devices, systems, associated components, and methods for holding and/or measuring and ultimately delivering a medical fluid to a patient. The present disclosure is generally described in connection with pressurized injection of a liquid radiological contrast material and/or a diluent/flushing agent in procedures such as angiography (CV), computed tomography (CT), ultrasound, magnetic resonance imaging (MRI), X-ray, and positron emission tomography (PET). However, it is to be understood that the devices, systems, and methods described herein may also be used in other applications where intravenous injection of precise doses of fluids such as contrast media, diluents, therapeutic agents, medicaments, drugs, and the like is indicated. Such procedures and/or treatments include but are not limited to nuclear medicine imaging, molecular imaging, radiopharmaceutical injection, and cardiovascular localized therapy treatment.

Further, although the present disclosure may be applied to computed tomography (CT) and/or angiographic-type injection systems, such as the MEDRAD® Stellant FLEX CT Injection System or the MEDRAD® Mark 7 Arterion Injection System, respectively, the present description is applied here in the context of injection systems designed for use in magnetic resonance (MR) imaging procedures. In particular, the present disclosure is described herein, by way of example, in connection with the MEDRAD® MRXperion MR Injection System offered by Bayer HealthCare LLC. Although the MEDRAD® MRXperion MR Injection System is a dual head (a/k/a dual syringe) type system, it will be apparent that the present disclosure may also be used in connection with single head (a/k/a single syringe) type injection systems. In further embodiments, the injection systems described herein may be used in connection with a hand-held syringe injection protocol.

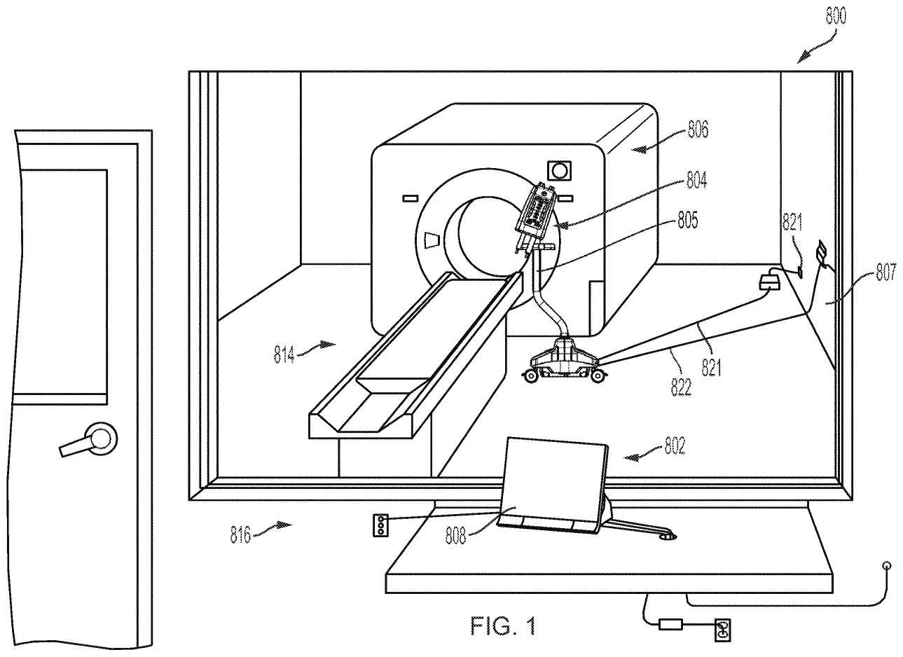

Referring first to FIG. 1, a non-limiting embodiment of a MR medical imaging suite 800, in which devices, systems, and methods in accordance with the present disclosure may be used is illustrated. Medical imaging suite 800 may be located in hospitals and/or imaging centers for performing imaging procedures on patients for diagnostic purposes. Medical imaging suite 800 may include scan room 814 in which fluid injection system 804 and medical imaging system 806 are located, and control room 816 from which an operator (e.g., a Radiologist) may conduct the injection and imaging procedures and monitor a patient through one or more workstation devices 802 associated with fluid injection system 804 and/or medical imaging system 806. Fluid injection system 804 may include a multi-fluid delivery system, such as the MEDRAD® MRXperion MR Injection System introduced above. As shown in FIG. 1, workstation device 802 may be used by the operator to set up and/or execute a fluid injection procedure. In some non-limiting embodiments, workstation device 802 may provide a user interface (e.g., an application-based user interface, a web-based user interface, etc.) on display unit 808 for control of fluid injection system 804 and medical imaging system 806. The parameters of an injection protocol of a fluid injection procedure may be inputted by the user, recalled from a common database, or automatically generated by a controller or an injection protocol management system associated with the fluid injection system 804, and, in some non-limiting embodiments, displayed on workstation device 802 and/or communicated to fluid injection system 804 for execution.

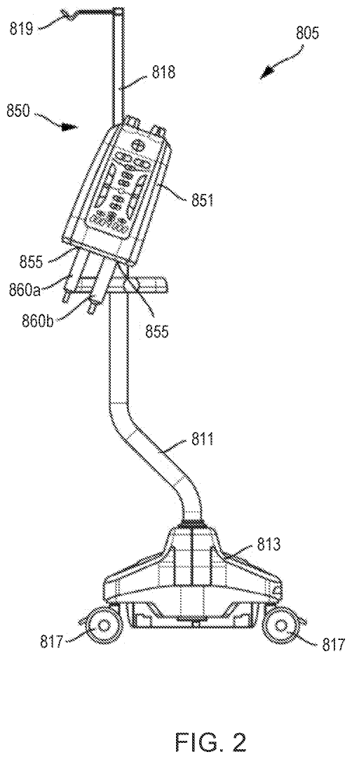

Referring now to FIG. 2, a diagram of a non-limiting embodiment of fluid injection system 804 shown in FIG. 1, of the type with which devices, systems, and methods in accordance with an aspect or example of the present disclosure may be used is illustrated. Specifically, FIG. 2 is an enlarged and partial view of a scan room unit 805 of the MEDRAD® MRXperion MR Injection System. While various embodiments of the present disclosure are described in reference to use of the MEDRAD® MRXperion MR Injection System, it is understood that the various systems and methods may be applied with other types and brands of fluid injection systems and that the systems and methods are not limited to a particular fluid injection system. In some non-limiting embodiments, scan room unit 805 includes pedestal 811 mounted to base 813 to which lockable casters 817 are affixed at bottom for use in moving the scan room unit 805 within scan room 814 as needed during imaging procedures. Pedestal 811 may also include an integrated IV pole 818 inclusive of one or more hooks 819 for hanging IV-related accessories. In some non-limiting embodiments, base 813 houses various electronic and communications components as well as the power supply for scan room unit 805. Among other connections, scan room unit 805 has, as shown in FIG. 1, a power cable 821 and a fiber optic link 822 in communication with workstation device 802 to enable control of the operation of the fluid injection system 804 from outside of scan room 814.

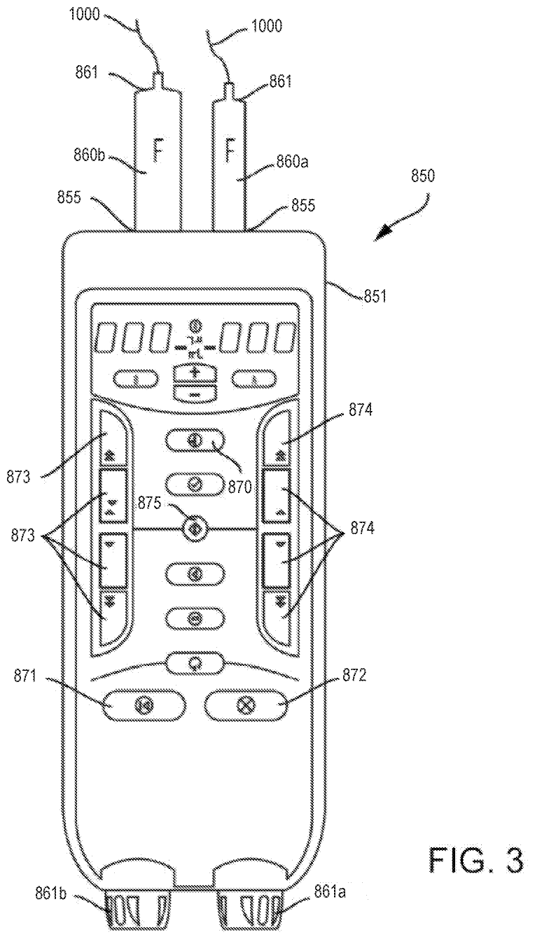

Referring now to FIG. 3 and with continued reference to FIG. 2, an enlarged view of an injector head unit 850 of fluid injection system 804 shown in FIG. 2 is shown. Specifically, FIGS. 2 and 3 illustrate a non-limiting embodiment of injector head 850 of scan room unit 805 of the fluid injection system 804. As these illustrations show, injector head 850 may include housing 851 and at least one fluid reservoir 860a/860b, such as at least one syringe. The injection system 804 may include a drive member to control fluid flow into or out of the fluid reservoir/syringe, such as a piston associated with each syringe 860a/860b that drives an associated plunger within a barrel of the syringe. The controller or injection protocol management system may control actuation of the drive member. Each of syringes 860a/860b is adapted to releasably interface with housing 851 at port 855 therefor and are configured to be filled with a medical fluid F, such as an imaging contrast media in syringe 860a, and a flushing fluid such as a saline solution in syringe 860b.

In some non-limiting embodiments, the fluid injection system 804 may be used during a conventional imaging procedure to inject the contrast media and/or saline into the vasculature of the patient by selectively driving the plunger associated with contrast syringe 860a and/or saline syringe 860b or both with their respective drive components. As shown in FIG. 3, for each syringe 860a/860b, the drive component may move the plunger toward the distal end 861 of the syringe to expel the fluid therein from the syringe into and through fluid path set 1000 during a priming, purging, and/or fluid delivery step. In some non-limiting embodiments, fluid path set 1000 may include at least one tube or tube set configured to be in fluid communication with each syringe 860a/860b to place the syringe(s) in fluid communication with a flexible administration tube and its associated catheter for delivering the fluid(s) from each or both syringes 860a/860b to the patient at the desired vascular access site.

As shown in FIG. 3, injector head 850 may include a variety of control buttons, knobs, and indicators through which to monitor and affect operation of fluid injection system 804.

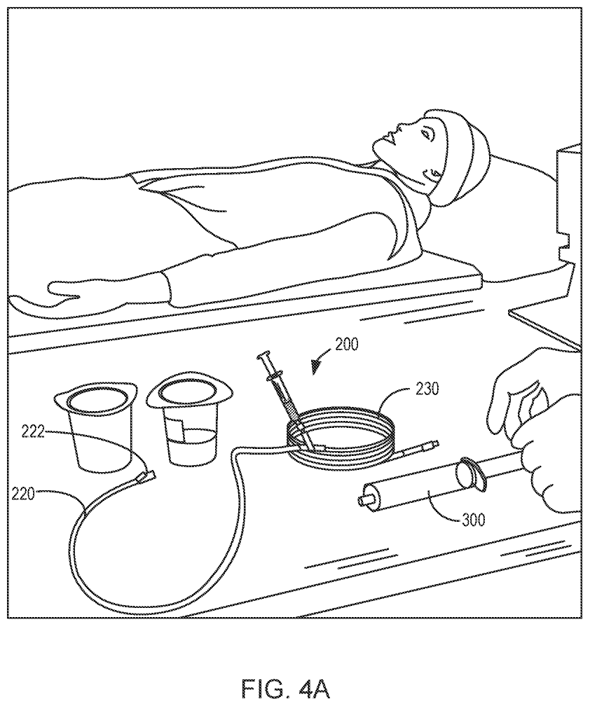

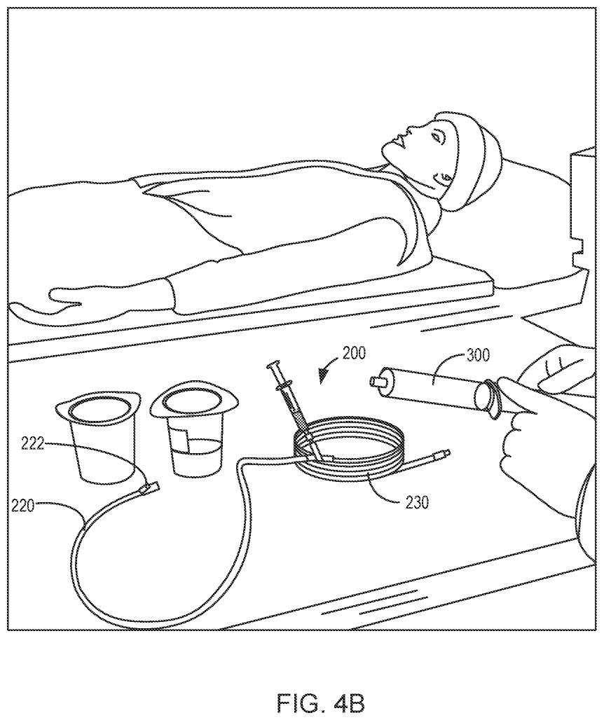

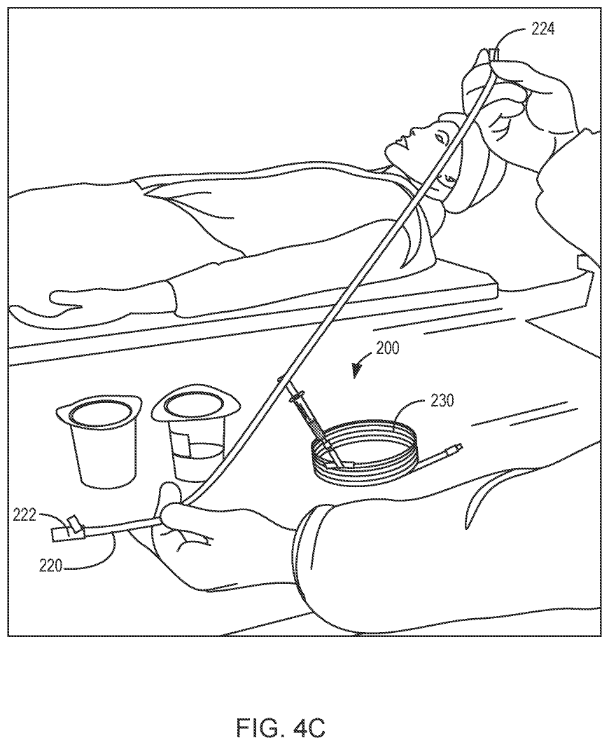

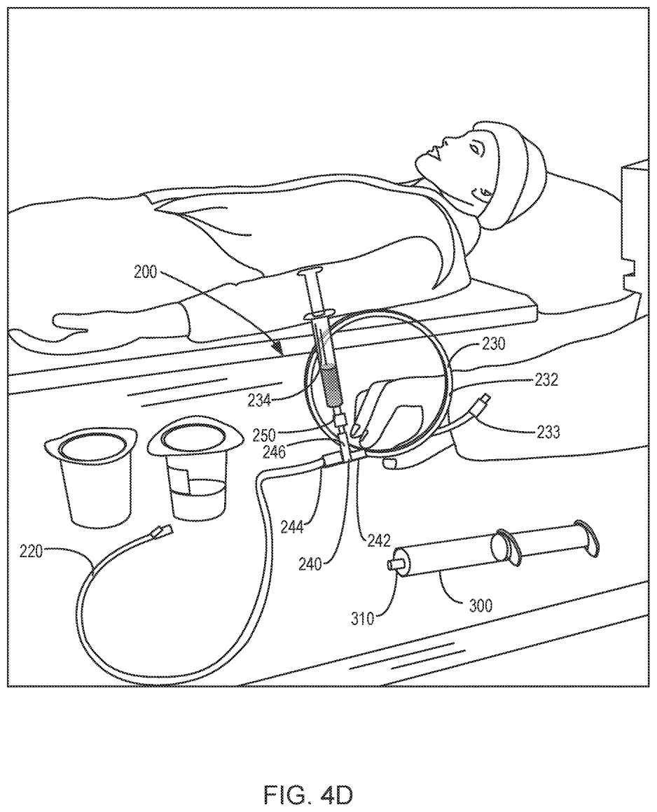

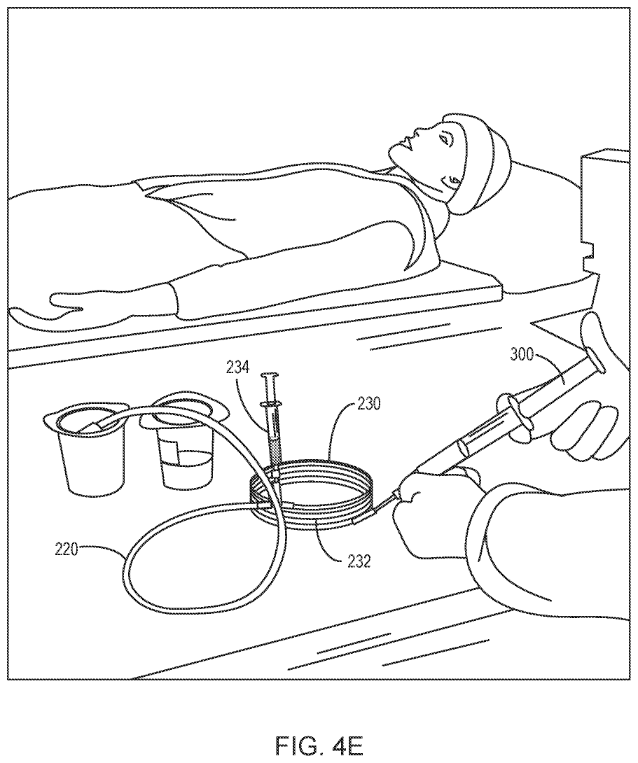

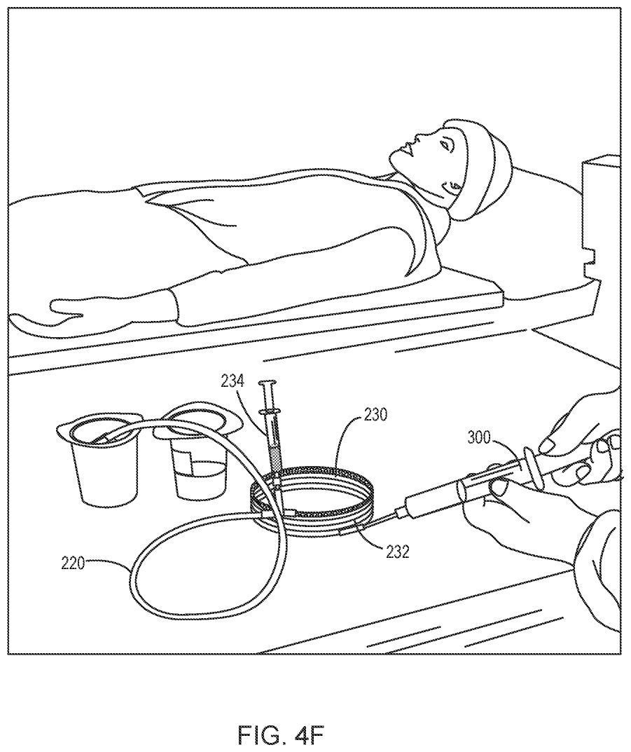

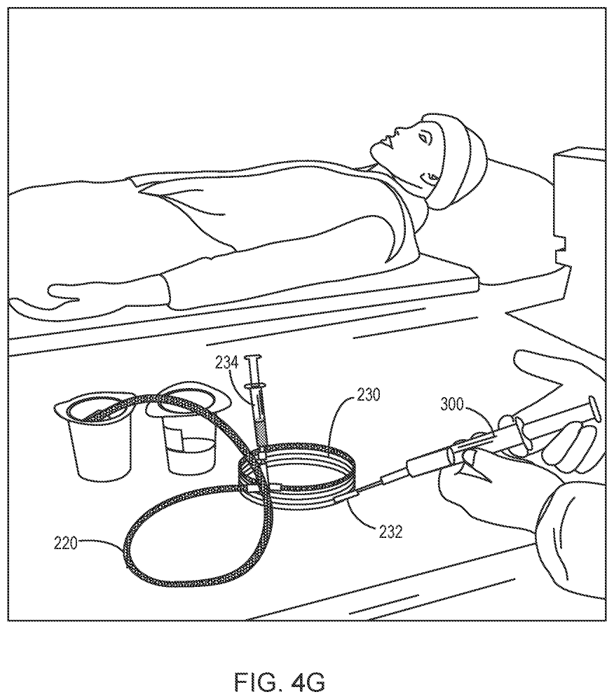

Referring now to FIGS. 4A-4G, implementations of a non-limiting embodiment of system 200 for use with a fluid injection system in accordance with an aspect or example of the present disclosure are illustrated. As shown in FIGS. 4A-4G, system 200 is applied, for illustrative purposes, to the fluid injection system 804 shown in FIGS. 1-3 or alternatively, to a hand-held syringe-based injection set up, as illustrated. FIG. 4A shows, in one non-limiting embodiment, the present system 200 for delivering precise, small volumes of a medical fluid, such as a contrast agent, to a patient. In some non-limiting embodiments, system 200 may include a combination of a single-use (per patient) tubing set 220 and a multi-use (per day) set 230. In other embodiments, the entire system 200 may be a configured as a single-use system to be discarded or recycled after the patient injection protocol is completed. The present system 200 may also include a syringe 300. Although the present disclosure requires use of a syringe, it should be apparent that one need not be supplied as part of a kit containing the components of the present disclosure, as such a syringe may be obtained commercially from a variety of suppliers. As shown In FIGS. 4A-4G, the syringe may be a hand-held syringe or in other embodiments, the syringe may be attached to a fluid injection system, as described herein. For example, the syringe may be attached to a single head or dual head fluid injection system, such as to a saline filled syringe attached to injector head 850.

Referring now to FIGS. 4B-4D, implementations of various components including syringe 300, single-use second tubing set 220, and multi-use first tubing set 230 of a non-limiting embodiment of system 200 of the present disclosure are illustrated. 4B shows the operator holding the syringe 300, FIG. 4C shows the operator holding the single-use tubing set 220, and FIG. 4D shows the operator holding the multi-use set 230. In certain non-limiting embodiment, the syringe 300 may be a hand-held syringe or, alternatively, may be configured for connection to the injector head 850 of the injection system 804 and, as such, may be implemented as saline or flushing fluid syringe 860b releasably attachable to injector head 850 at the designated port 855 therefor. In other embodiments, the flushing fluid may be actuated and caused to flow by a peristaltic pump assembly. Preferably implemented as a low-pressure tubing set, the single-use tubing set 220 has a one-way check valve 222, for example at its distal end, which is intended for connection to the catheter that is to be inserted into the appropriate vessel of the patient. Check valve 222 may prevent backflow through the single-use components and contamination of upstream multi-use components with bodily fluid and/or microbial contaminants. At its proximal end, the single-use tubing set 220 is intended to be connected to the distal end of the multi-use set 230. The multi-use set 230 at its proximal end is adapted to be connected to the syringe 300, for example, as implemented as saline syringe 860b of the injection system 804 shown in FIGS. 2 and 3.

As shown in FIG. 4D, the multi-use set 230 includes a first tubing set 230 having a first length of tubing 232, a prefilled syringe 234, a three-port stopcock valve assembly 240 and a swabable valve 250, such as a swabable valve commercially available from Halkey-Roberts, St. Petersburg, Florida. Although illustrated in FIG. 4D as a prefilled syringe containing an MR contrast agent, as an alternative, the prefilled syringe 234 can be implemented in a different form such as a bag, a pouch or other suitable holder of MR contrast media such as a vial of MR contrast to which a spike on the valve assembly 240 may be connected. The first length of tubing 232 may have an internal diameter larger than, equal to, or smaller than that of the single-use tubing set 220. At its proximal end, the tubing section 232 is designed to connect via a luer or other suitable connector 233 to the nozzle 310 of syringe 300/860b. The three-port valve assembly 240 features a proximal port 242, a distal port 244 and a third port 246. The proximal port 242 connects via a second connector member, such as a luer or other suitable connector to the distal end of first length of tubing 232, and the distal port 244 is designed to connect to the proximal end of single-use tubing set 220 via a second connector member, such as a luer or other suitable connector 224, as illustrated in FIG. 4C. Third port 246 is designed to connect to the fourth connector member via the swabable valve 250 or other suitable dripless valve assembly, with the other end of the swabable valve 250 intended for connection to the medical fluid container, such as the luer tip of prefilled syringe 234. In this non-limiting embodiment, the prefilled syringe 234 is filled with MR contrast media. The syringe 234 may include indicia on an outer surface thereof, indicating a volume of contrast media contained therein and allowing the user to determine the volume of contrast media to be injected into the first length of tubing, as described herein. In certain embodiments, syringe 234 may be a hand-held syringe and include one or more stops or threading, for example on a piston of syringe 234, which allows for precise administration, by moving an desired number of stops or dialing a correct number of threads, of accurate transfer of small volumes of contrast media from syringe 234 into the first length of tubing 232. Alternatively, syringe 234 may be an exact dose syringe, such as a micro dose syringe, having the exact desired volume of contrast agent to be used in a single injection protocol, such that the exact volume of contrast in injected from syringe 234 into the first length of tubing 232.

The present system 200 is suited to be connected to and operated according to an exemplary method with a fluid injection system 804 as described herein. For example, with its proximal end connected to the appropriate port 855 of the injector head 850, the syringe 300/860b may be connected by its nozzle 310 to the first length of tubing 232 of multi-use set 230 via luer or other suitable connector 233. At the other end of multi-use set 230, the distal port 244 of valve assembly 240 may be connected by a third connector member to the proximal end of single-use tubing set 220 via luer or other suitable connector 224. Assembled in this manner, the present system 200 would be ready for use with fluid injection system 804 with an injection procedure of a contrast enhanced imaging procedure.

Prior to it being connected to the multi-use tubing set 230, the syringe 300/860b according to the present embodiment of the method may be primed with saline from a bag, bottle or other suitable container. With its nozzle 310 connected to the container, the syringe 300/860b may be filled with saline or other flushing fluid and any air primed out of the syringe either manually or automatically using fluid injection system 804. Once primed, the saline syringe 300/860b is ready to be connected to the first connector member at the proximal end of first length of tubing 232 of multi-use set 230 as described above. With the multi-use set 230 connected to the single-use tubing set 220 as described herein, the saline syringe 300/860b is ready to be used to prime both the first length of tubing 232 of multi-use set 230 and the single-use second tubing set 220, as shown in FIG. 4E. During the priming of first length of tubing 232 and single-use second tubing set 220, no saline will flow into prefilled syringe 234 (or other suitable container 234) as valve assembly 240 is configured to prevent fluid communication with the third port. In various embodiments, the prefilled syringe 234 does not need to be connected to the swabable valve 250 while tubing section 232 and single-use tubing set 220 are being primed with saline. In various embodiments, a one-way valve within swabable valve 250 may prevent saline from entering the contrast syringe 234 and thus, prevent dilution of the contrast media contained therein.

Once the priming of syringe 300/860b, first length of tubing 232 and single-use second tubing set 220 with saline is complete, the present system 200 may be filled with the small, accurate volume of MR contrast media according to the present method. In particular, tubing section 232 of multi-use set 230 may be primed with the contrast. To that end, the syringe 234 may be attached to swabable valve 250 and valve assembly 240 may be turned to provide fluid communication between the third port 246 and the proximal port 242 of the valve assembly. The fluid injection system 804 can be controlled via its programming, its control buttons, or its manual knob 861b to retract the plunger of syringe 300/860b and thereby draw the desired volume of MR contrast from prefilled syringe 234 (or other suitable container 234) through the swabable valve 250, through both third port 246 and proximal port 242 of valve assembly 240 and into first length of tubing 232. As the MR contrast media is drawn into tubing section 232, fluid communication with distal port 244 is blocked by valve assembly 240 preventing the saline in single-use tubing set 220 from being drawn upstream as well thereby keeping the single-use tubing set 220 primed with saline. As illustrated in FIG. 4F, first length of tubing 232 filled with a desired volume of contrast agent at its distal end with, for example in one embodiment, 5 ml of MR contrast, with its remaining upstream portion of the first tubing set, and the entirety of single-use tubing set 220, still primed with saline.

Once the present system 200 is filled with both saline flushing volume and the desired amount of MR contrast as described herein, the system 200 is ready to be used according to an exemplary method with fluid injection system 804 to carry out the desired injection procedure. For example, in certain embodiments, the valve assembly 240 may be configured to provide fluid communication between the proximal port 242 and the distal port 244 and the fluid injection system 804 can be controlled via its programming, its control buttons, and/or its manual knob 861b to extend the plunger further into the barrel of saline syringe 300/860b and thereby pressurize the fluid within the fluid path defined by syringe 300/860b, tubing section 232, valve assembly 240, single-use tubing set 220 and the catheter to which the single-use tubing set 220 has been connected. In this manner, the desired amount of MR contrast will travel from tubing section 232 through valve assembly 240, single-use tubing set 220 and, ultimately, into the patient via the catheter to which the single-use tubing set 220 has been connected. This is illustrated in FIG. 4G, which shows the desired amount of contrast being pushed from tubing section 232 of multi-use tubing set 230 through single-use tubing set 220 and into the patient via the catheter (not shown) that has been inserted into the desired vessel (note, while FIG. 4G illustrates the distal end of tubing section 220 draining into a container, in use, the distal end of tubing section 220 is connected to a catheter fluidly connected to a patient's vasculature). According to certain embodiments, the controller of the fluid injection system may be configured to actuate an actuator to control the valve assembly 240 to rotate the associated stopcock between the various desired positions according to whether fluid communication is desired between the third port 246 and the proximal port 242 or between the proximal port 242 and the distal port 244, or to prevent fluid communication between any of the three ports, for example to stop an injection protocol. In other embodiments, one or more one-way check valves or high crack pressure valves may be utilized to control flow of contrast and saline through the proximal port 242, the distal port 244, and/or the third port 246 during priming and injection processes.

One intent of system 200 is to use the multi-use first length of tubing 232 as a short-term volume retention vessel for the small volumes of contrast to be delivered to the patient. As described herein, the contrast can be deployed in the form of a prefilled syringe, a bag, a pouch, or a vial to which a spike with a male luer may be connected to the multi-use tubing set 230 though valve assembly 240. For example, the third port 246 of valve assembly 240 will prevent any pressurized saline from syringe 300/860b from entering the prefilled syringe (or bag, pouch, or vial) and allow for KVO (keep vein open) operation and a saline flush by fluid communication between the proximal port 242 and the distal port 244. The proximal port 242 of valve assembly 240 will allow the contrast to be drawn into the tubing section 232 of multi-use tubing set 230 and “charge the line” upon retraction of the plunger of saline syringe 300/860b. It should be apparent that in certain embodiments, tubing section 232 may be implemented with a larger interior diameter (ID) and/or length so that it can hold any volume of contrast between 1-30 ml or even more. Alternatively, a smaller ID for tubing section 232 may allow for smaller, more precise volumes of contrast to be held therein. Once first length of tubing 232 is filled with the desired amount of contrast, the plunger of syringe 300/860b can be advanced to flush the system with saline and drive the contrast into the single-use tubing set 220 and ultimately into the patient via the catheter. This extension of the plunger into the barrel of syringe 300/860b also flushes the system and the vessel of the patient with saline allowing the contrast to be taken by the vasculature of the patient. Although primarily described herein in the context of the delivery of contrast, the present system 200 is equally well suited for use in the delivery of medications to a patient.

Whether used with a single syringe or dual syringe type injection system, embodiments of the present system 200 with the multi-use set 230 and swabable value 250 may be used either with a prefilled syringe 234 or a bag, a pouch or a vial of contrast or medications into which a spike has been inserted to access multiple doses of contrast media for multiple injection protocols. In various embodiments, the fluid injection system 200 can be controlled from the scan room such as scan room 814 of an MR suite. The injection system 200 can also employ a wireless remote so its operator can remotely control the injection procedure and have patient safety in mind. The system is also readily adaptable for use with both syringes of a dual head injection system. For example, in addition to its use of a saline syringe as described above, the present system may also use the contrast syringe of a dual head injection system to draw the desired amount of contrast (or medication) or more into the tubing section 232 of the multi-use set 230. In this regard, the contrast syringe chosen may have a smaller diameter than that of the saline syringe. Lastly, the present system is also readily adaptable for use with a cassette-type injection system in lieu of a syringe-based injection system. In this regard, the present system can be implemented as a cassette designed to be loaded within and operated by such a cassette-type injection system. One or more stopcocks can be employed to, for example, allow a continuous supply of saline to the primary syringe (i.e., for filling and re-filling same).

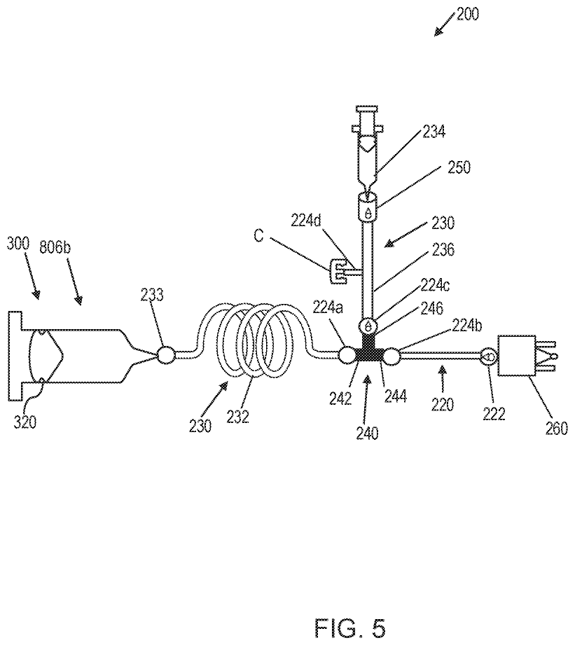

Referring now to FIGS. 5-12, schematics of various non-limiting embodiments of system 200 in accordance with an aspect of the present disclosure are illustrated. The exemplary embodiments of system 200 in FIGS. 5-12 may be the same or similar to system 200 shown in FIGS. 4A-4G, in which like reference characters refer to like parts or components.

With references to FIGS. 5, 6, 8A, and 8B, exemplary embodiments of system 200 for use with a fluid injection system are shown including first tubing set 230, which may further include first length of tubing 232 and second length of tubing 236. As discussed above, first tubing set 230 may be multi-use set 230, such that set 230 maintains sterility and is suitable for multiple injection procedures for multiple patients without contamination of the multi-use components. The first length of tubing 232 may be sized to hold a volume of 30 mL or less of fluid. For certain patients and/or use cases, first length of tubing 232 may be sized to a hold a volume in the inclusive range of 0.1 mL and 15 mL of fluid. However, the appropriate sizing and volumetric constraints of the tubing ultimately depend on the use case in which system 200 is applied, such as the treatment indicated and individual patient needs. Accordingly, the first length of tubing and/or tubing set 230 may also be sized and configured to hold a greater or lesser volume of fluid. For example, first length of tubing 232 may be sized to a hold a volume in the inclusive range of 0.1 mL to 5 mL of fluid or even ranges of 0.1 mL to 0.5 mL of fluid for imaging procedures of pediatric patients.

In some non-limiting embodiments, first length of tubing 232 of multi-use set 230 may be manufactured of a translucent polymeric material such that a fluid in the first length of tubing is visible to a user and/or a sensor of a fluid injection system 804. This visibility primarily serves to allow for accurate measurement of the medical fluid F as it is drawn from syringe 234 into system 200 and/or is held in the multi-use set 230. It is further considered that virtually any tubing of system 200 including but not limited to multi-use set 230 and single-use set 220 may also be made of translucent or clear material. The inclusion of translucent tubing provides additional configurations and opportunities to hold and/or measure medical fluid prior to injection. Further, additional safeguards are provided by allowing a user and/or sensors of a fluid injection system to ensure proper functionality of the system is maintained. To further aid a user or fluid injection system with the measurement of fluid, the first length of tubing 232 (or other suitable tubing portions of the system), may include indicia, such as graduation volume demarcations, configured to provide volumetric measurements of the fluid within first tubing portion 232 or within the other given length of tubing. The indicia may be read by a user or by one or more sensor in communication with the controller of the injection system, to begin or stop a priming or fluid delivery step according to a method of using system 200 based of the volume of the medical fluid drawn into the first length of tubing 232.

In some non-limiting embodiments, where system 200 is applied with a fluid injection system such as fluid injection system 804, the fluid injection system may include at least one measurement sensor configured to measure the volume of fluid, such as liquid contrast media, that is drawn into the first length of tubing 232. The measurement sensor may be configured to communication with a controller of the fluid injection system when a predetermined volume of liquid contrast media is drawn or pushed into the first length of tubing.

FIGS. 5, 6, 8A, and 8B further show first connector member 233, which may be positioned at the proximal end of first length of tubing 232. First connector member 233 may be configured to connect to syringe 300 which may be intended for connection to fluid injection system 804. Accordingly, in some non-limiting embodiments of system 200, first connector member 233 may connect syringe 300/860b that may be a manual syringe, an auto-injector, or a syringe of a fluid injection system.

With continued reference to FIGS. 5, 6, 8A, and 8B, first length of tubing 232 and second length of tubing 236 may include valve assembly 240 disposed there between, such that valve assembly 240 is positioned at the distal end of first length of tubing 232. Valve assembly 240 may include a proximal port 242, a distal port 244, and a third port 246. As shown in the exemplary embodiment, second connector member 224a, which may be connected to proximal port 242, may further connect the distal end of first length of tubing 232 to proximal port 242. As further shown, third connector member 224b may be connected to distal port 244, and fourth connector member 224c connect a first swabable valve 250 and second length of tubing 236 to third port 246. As mentioned above, third port 246 is designed to connect to a swabable valve 250, and the connection may be made by third connector member 224c. In some non-limiting embodiments, as mentioned above, one or more of first connector member 233, second connector member 224 a, third connector member 224b, and fourth connector member 224c may include a one-way check valve and/or a luer connector, or a combination thereof. In some non-limiting embodiments, the connector members may be contained within or may be a part of a valve, inclusive of the connector members associated with valve assembly 240.

According to exemplary embodiments, container 234 containing a medical fluid F such as contrast media, may be removably connected to first swabable valve 250. Once the associated components of system 200 have been assembled, valve assembly 240 may selectively provide a fluid connection between proximal port 242, distal port 244, and third port 246, such that priming, purging, and/or fluid measurement and delivery may be executed using system 200. In various embodiments, valve assembly 240 may be manually actuated or actuated by an actuator in response to one or more signals sent from the controller of the fluid injection system. For example, in non-limiting embodiments where syringe 300/860b is provided containing a flushing agent such as saline, valve assembly 240 provides a fluid connection between proximal port 242 and distal port 244. Actuation of plunger 320 by the fluid injection system, then pushes the diluent through second tubing set 220 to flush administration line 220. In certain embodiments, actuation of plunger 320 may be manual actuation where the syringe 300/860b is a manual syringe.

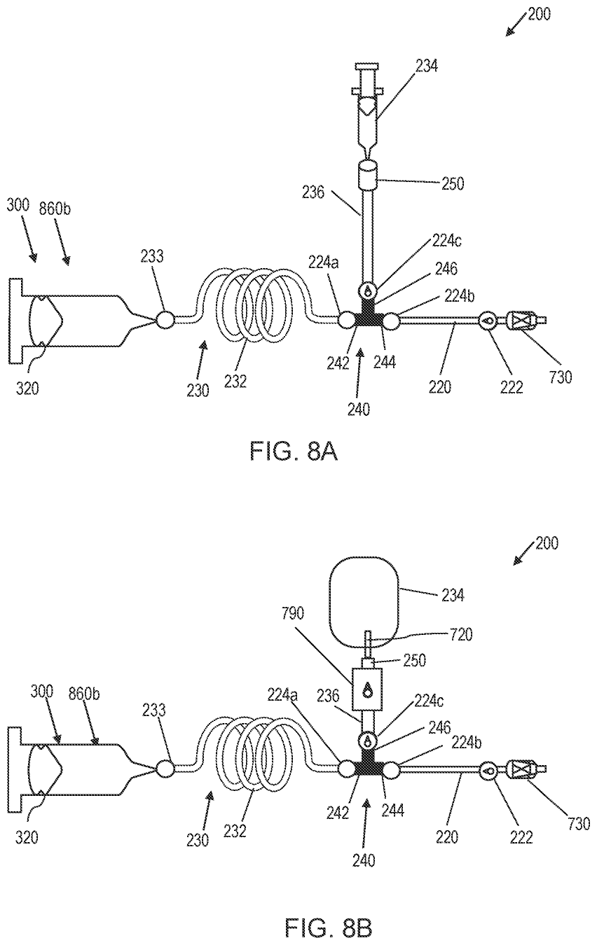

With reference to FIGS. 8A and 8B, non-limiting embodiments of system 200 are associated with a fluid injection system such as fluid injection system 804, and connector member 233 is connected to a syringe 300/860b of the fluid injection system, the actuation of the fluid injection procedure may be piston driven. In such embodiments, the fluid injection system 804 may be programmed or controlled to actuate the valve assembly 240 and/or a piston associated with the syringe 300/860b to retract plunger 320 to draw a predetermined volume of fluid into the first length of tubing 232 from the container 234. In other embodiments, syringe 234 may be a manual syringe that can manually add a desired volume of contrast fluid into the first length of tubing 232. For example, syringe 234 may include one or more stops associated with a defined volume of fluid, e.g., 0.1 mL or 0.5 mL, and the user may move the piston of syringe 234 the number of stops associated with the desired volume of contrast agent for a procedure. In other embodiments, syringe or contain 234 may include a dial, such as a threaded plunger, where a distance of turning of the dial is associated with a volume of contrast, and the user may rotate or otherwise move the dial the number of movements associated with the desired volume of contrast agent for a procedure. Other methods of accurately dispensing the desired volume of contrast from container 234 are contemplated.

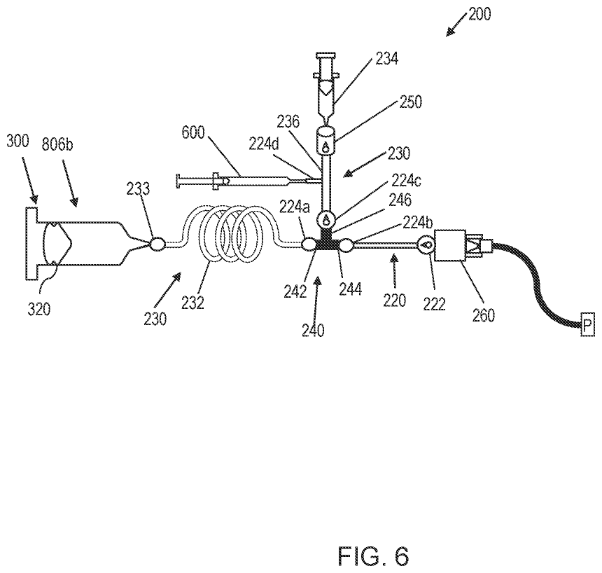

In some embodiments, as shown in FIGS. 5 and 6, third connector member 224b of system 200 may connect a proximal end of second tubing set 220 to distal port 244 of valve assembly 240. In some non-limiting embodiments, second tubing set 220 may be an administration line or single-use set 220, as discussed herein. Tubing set 220 may include one-way check valve 222 positioned therein, for example at the distal end of tubing set 220 to allow fluid to flow in a single direction therefrom, i.e., fluid flow is directed toward the patient and proximal fluid flow is prevented to prevent contamination of the multi-patient portions of the system. The distal end of tubing set 220 may further be connected to a catheter, such as catheter 260, or another suitable connector to a patient line, for delivery of the medical fluid F to a patient.

In some non-limiting embodiments, such as those shown in FIGS. 5, 6, and 8A, container 234 containing the medical fluid F may be a second syringe 234. In such exemplary embodiments, the valve assembly 240 may provide a fluid connection between third port 246 and proximal port 242. The second syringe 234 may then selectively control fluid flow between second syringe 234 and first length of tubing 232, such that a measured volume of fluid is allowed to flow from second syringe 234 to the first length of tubing. Similar to syringe 300/860b, second syringe 234 may be a manual syringe, or in embodiments where the system 200 is associated with a fluid injection system, second syringe 234 may be a syringe connected to and operated by the fluid injection system.

In some non-limiting embodiments, as illustrated in FIGS. 5 and 6, system 200 may include an additional syringe such as third syringe, such as micro syringe 600. To accommodate micro syringe 600, an additional connector member 224d may be provided between the third port 246 and the syringe 234. As shown in FIG. 5, micro syringe 600 may be removably connected to the additional connector member 224d and the third port 246. In such an embodiment, medical fluid may be drawn from container 234 by syringe 300/650b in a priming or injection procedure. For example, when precise small volumes of medical fluid are to be drawn from syringe 234, micro syringe 600 may be used to accurately measure out the desired volume from syringe 234 and then used to inject the desired volume into the length of tubing 232 via valve assembly 240. When use of micro syringe 600 is not desired or indicated for a particular procedure, the micro syringe 600 may be disconnected from additional connector member 224d, and the additional connector member may receive a cap C.

The medical fluid may also be pushed from container 234 in embodiments where container 234 is implemented as a syringe, as shown in FIG. 5. Alternatively, container 234 may be disconnected from system 200 and swabable valve 250 may receive a cap to maintain pressure of system 200. In such embodiments, the micro syringe 600 may provide the medical fluid for a priming and/or injection procedure such that a measured volume of medical fluid is allowed to flow from the micro syringe 600 to the first length of tubing 232. As shown in FIG. 6, where micro syringe 600 and container 234 are both provided, the connector members 224a/224b/224c and the additional connector member 224d are configured to selectively control fluid flow between the container 234, the micro syringe 600, and the first length of tubing 232. Thereby, a measured volume of medical fluid is allowed to flow from the container 234 to the first length of tubing 232.

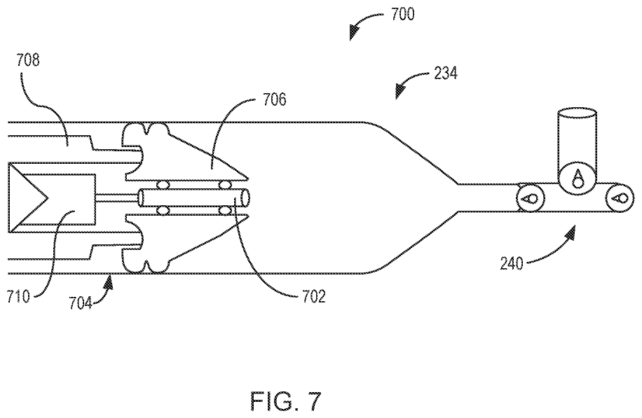

Referring now to FIG. 7, an exemplary embodiment in which second syringe 234 is a pulsing syringe 700 is illustrated. Pulsing syringe 700 may include a dispenser piston 702 within the barrel of pulsing syringe 700 for dispensing a controlled and accurate low dose of medical fluid. Dispenser piston 702 may be positioned on a distal end of a plunger assembly 704 of the pulsing syringe. Dispenser piston 702 may also be surrounded by a cover 706 of plunger assembly 704. Cover 706 may be made of rubber or another suitable elastomer. Pulsing syringe 700 may further include solenoid 708, the actuation of which may drive an engagement piston 710. The engagement piston 710 thereby engages the rubber cover 706 of the plunger assembly, thereby dispensing a controlled dose of medical fluid.

Accordingly, pulsing syringe 700 is configured to selectively control fluid flow from the second syringe 234 to the first length of tubing 232, through valve assembly 240, by delivering a predetermined volume of medical fluid a predetermined number of times. The exemplary non-limiting embodiment of pulsing syringe 700 may be associated with fluid injection system 804 described herein. The fluid injection system 804 may be programmed or controlled to actuate the solenoid 708 and thereby the dispensing piston 702 to deliver the predetermined volume of medical fluid the predetermined number of times (pulses). In some non-limiting embodiments, the predetermined volume of medical fluid may be as little as 0.1 mL per pulse or as much as 1 mL per pulse. It is considered that the predetermined volume could be any volume for which the system 200 has volumetric capacity. The predetermined number of pulses could likewise be any number of pulses for which the system 200 has capacity. For example, to achieve a volume of 5 mL of medical fluid held in system 200, pulsing syringe may pulse 5 times while dispensing 1 mL per pulse.

In some non-limiting embodiments, as shown in FIG. 8B system 200 may further include dial 790 positioned between container 234 and the first length of tubing 232. As the valve assembly 240 provides a fluid connection between the third port 246 and the proximal port 242, the dial 790 may selectively control fluid flow between the container 234 and the first length of tubing 232, allowing a precise measured volume of medical fluid to flow from the container to the first length of tubing. Dial 790 may be configured to only allow a predetermined volume of fluid to be drawn from the container 234 per rotation or per “click” (partial rotation or linear movement increment, for example denoted by an audible or tactile click) of the dial.

Referring still to FIG. 8B, container 234 containing the medical fluid is pouch, a bag, a bottle, or another vessel may be configured to receive a spike. System 200 may include spike 720 connected to swabable valve 250 of the third port 246. The container 234, shown in FIG. 8B as a fluid bag, receives spike 720 and is fluidly connected to the valve assembly 240. In such exemplary embodiments, the fluid may be drawn from container 234 by the syringe 300/650b, or by another means as described herein. For example, as shown in FIG. 6, system 200 may include the micro-syringe 600 connected to additional connector member 224d. In the embodiment shown in FIG. 8B, the dial 790 may be used to draw the medical fluid from container 234. In other embodiments, another syringe or device may be provided to draw or push the medical fluid from the container 234.

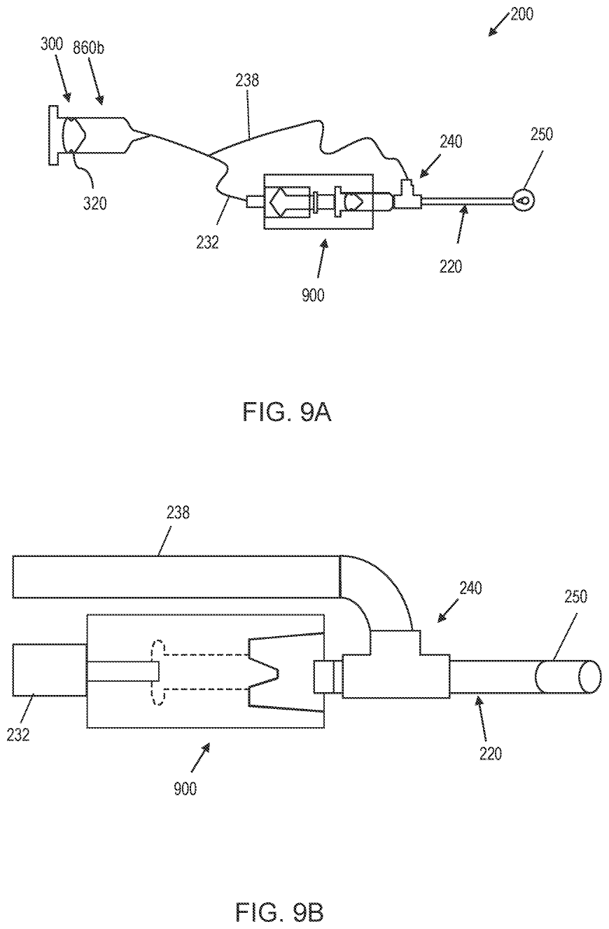

Referring now to FIGS. 9A-11, non-liming embodiments of system 200 in which the system includes a saline bypass line, illustrated as third length of tubing 238 are shown. In the exemplary embodiments, syringe 860 may be a dual syringe system such as that of fluid injection system 804. In such embodiments, the container containing medical fluid may be a pre-filled component 900 positioned between the first length of tubing and the valve assembly 240. As shown in FIG. 9A, the proximal end of the third length of tubing 238 is connected to the syringe 860b, and the distal end of the third length of tubing is connected to the second tubing set 220 at valve assembly 240. In these exemplary embodiments, the bypass line 238 and the first length of tubing 232 are in parallel, and both may ultimately deliver fluid to a patient through the administration line such as second set 220.

The bypass line 238 is configured to deliver a flushing fluid to the second tubing set 220 and to the patient. Upon actuation of the piston/plunger 320 of the syringe 300/860b, the diluent is delivered through the first length of tubing, actuating the pre-filled component 900. Actuation of the pre-filled component 900 pushes the medical fluid through the first length of tubing to the second tubing set 220 and ultimately to the patient. Implementation of a saline bypass line such as bypass line 238 in system 200 provides the advantages of avoiding fluid communication between multiple medical fluids such as a diluent and a medicament. For example, avoidance of fluid communication between saline and contrast media prevents unwanted dilution of the contrast media, which may also allow lower volumes of contrast media.

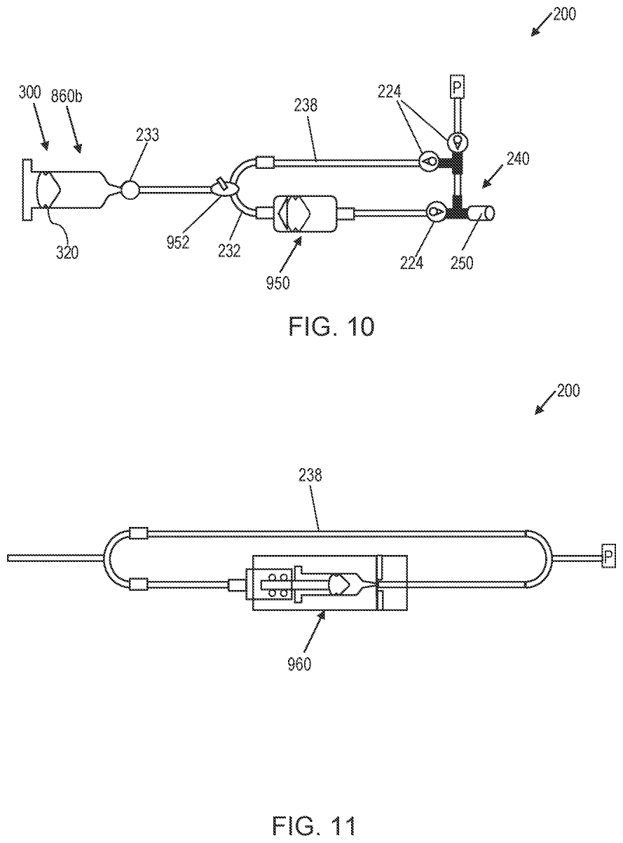

With reference to FIG. 10, system 202 may also include a three port valve positioned between connector member 233 and the distal end of each the bypass line 238 and the first length of tubing 232 to control the fluid flow from the syringe 300/860b to bypass line 238 and/or to first length of tubing 232. As shown in FIG. 10, the three port valve may be stopcock 952, which may be associated with fluid injection system 804. Fluid injection system 804 may be configured to actuate the stopcock to complete a fluid connection between the syringe 300/860b and either first length of tubing 232 and the pre-filled component (i.e., two-sided plunger 950 of FIG. 10), or bypass line 238. Stopcocks associated with fluid injection system 804 may also be implemented in place of other valves/valve assemblies of the present disclosure such that system 200 may be at least partially or fully automated.

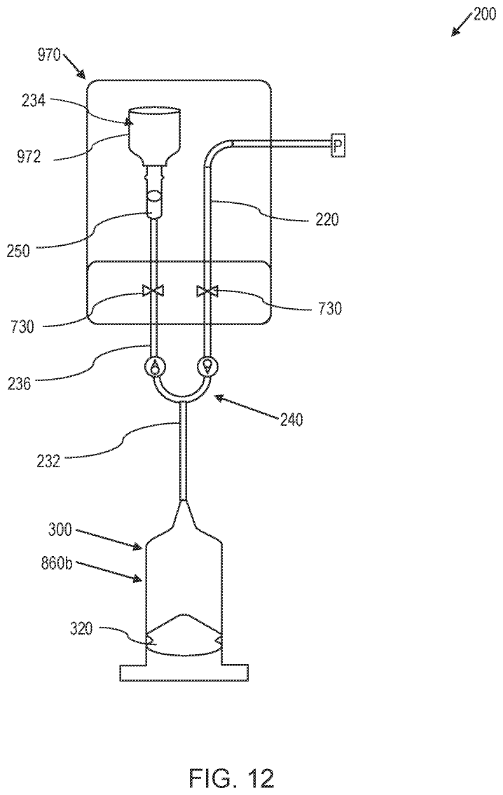

In some non-limiting embodiments, the container 234 containing the medical fluid may be any suitable pre-filled component such as a syringe with a two-sided plunger 950 as shown in FIG. 10, a rolling diaphragm 960 as shown in FIG. 11, or a cassette 970 as shown in FIG. 12. The prefilled component may be manufactured with a precise does of medical fluid such as contrast or may be filled by a user prior to implementation in system 200. Use of a pre-filled component such as a two-side plunger 950, rolling diaphragm 960, cassette 970, etc. is ideal for embodiments of system 200 applied to fluid injection system 804. In some embodiments, the pre-filled component may include computer readable media such as a barcode, QR code, RFID tag, text, symbol, or the like, and the fluid injection system may include a scanner or other sensor capable of reading the barcode and conveying data about the pre-filled component to the controller or injection protocol management system associated with the fluid injection system 804. The controller may use the data in conjunction with other input data to execute the appropriate priming/injection procedure. Implementation of a pre-filled component such as those shown in FIGS. 9A-12 allows for using a smaller amount of tubing when providing larger volumes of medical fluid. Instead of providing an extended length of tubing, a pre-filled component may be provided to take up less linear space in system 200.

Referring now to FIG. 12, a non-limiting embodiment of system 200 is shown including a pre-filled component in which the pre-filled component is implemented as a cassette 970 containing a pre-filled bottle or syringe 972. In the present exemplary embodiment, system 200 may be the same as or similar to the embodiments exemplified in FIGS. 5 and 6 and may further include air sensors 730. Air sensors 730 may be positioned on second length of tubing 236 and/or on administration line 220. The air sensors may be configured to detect air in the system 200 and may communicate the detection of air with the controller of the fluid injection system. When air is detected in system 200, the fluid injection system may be controlled to begin a flushing procedure or to stop a priming or injection procedure.

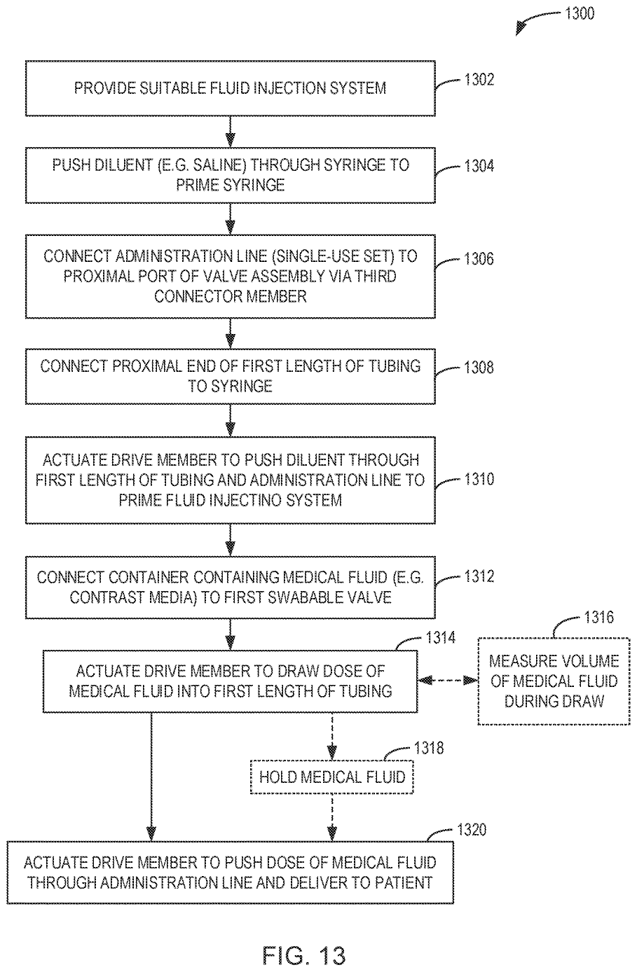

Referring now to FIG. 13, a sequence diagram of a method 1300 for using system 200 applied to fluid injection system 804 is illustrated. In some non-limiting embodiments, a method step 1302 for using a fluid injection system may include providing a suitable fluid injection system and the various desired components of system 200, such as those discussed above in detail.

For exemplary purposes, a fluid injection system may include a housing, at least one syringe, at least one drive member, and at least one controller programmed or configured to control actuation of the at least one drive member, and an administration line according to any of the various embodiments of the system described herein. The fluid injection system may further include a first length of tubing having a proximal end and a distal end, and a first connector member positioned at the proximal end of the first length of tubing. The first connector member may be configured to connect to the at least one syringe of the fluid injection system, and a valve assembly may be positioned at the distal end of the first length of tubing. The valve assembly may include a proximal port, a distal port, and a third port. A second connector member may connect the distal end of the first length of tubing to the proximal port. A third connector member may be connected to the distal port and may be configured to connect to a proximal end of the administration line. A fourth connector member may connect a first swabable valve to the third port.

The fluid injection system may be provided in any suitable environment for carrying out the desired injection procedure or treatment. For example, if the desired procedure is injection of contrast media prior to an imaging procedure, the fluid injection system may be provided in medical imaging suite 800 discussed above.

At method step 1304 as shown in FIG. 13, the method may further include priming the at least one syringe by pushing a diluent through the at least one syringe. At method step 1306, the method may include connecting the administration line to the third connector member and to the proximal port of the valve assembly. At method step 1308, the method may further include connecting the proximal end of the first length of tubing to the at least one syringe of the fluid injection system. In this state, after the connections in method steps 1306 and 1308 have been completed, the fluid injection system may be ready to execute a saline priming procedure. Accordingly, at method step 1310, the method may include priming the fluid injection system by actuating the at least one drive member and pushing a flushing fluid, such as saline, from the at least one syringe through the first length of tubing and through the administration line.

As shown at method step 1312 of FIG. 13, the method may include connecting a container containing a medical fluid, such as MR contrast media, to the first swabable valve. However, it is to be understood that the connection of the container, which may be a syringe, a bag, a bottle, a cassette, or any other suitable container, may also be made prior to execution of the saline priming procedure of method step 1310, depending on the container implemented and the desired injection procedure. Once the fluid injection system is primed and the desired medical fluid is introduced to the system, the at least one drive member may be actuated to draw a desired dose or volume of medical fluid into the first length of tubing, as shown at method step 1314.

In some non-limiting embodiments such as shown at method step 1316, the contrast media or other medical fluid may be measured by the various means discussed above as the fluid enters the first length of tubing. Additionally or alternatively, at method step 1318, the medical fluid may be held within the system until execution of an injection procedure. In some non-limiting embodiments, the fluid may additionally or alternatively be measure while it is being held by the system. At method step 1320, when injection is desired, the at least one drive member may be actuated to push the dose of medical fluid from the first length of tubing through the administration line and to the patient.

Examples of various use cases for the present system include adaption to the imaging modalities of, for example, magnetic resonance imaging (MRI), nuclear medicine (including positron emission tomography (PET)), computed tomography (CT), and angiography. Three such examples are provided below.

While several examples of the devices, systems, associated components, and methods for measuring precise doses and/or holding precise doses of medical fluid for use in fluid injection systems are shown in the accompanying figures and described in detail hereinabove, other aspects will be apparent to, and readily made by, those skilled in the art without departing from the scope and spirit of the disclosure. Accordingly, the foregoing description is intended to be illustrative rather than restrictive. The various embodiments of the present disclosure described hereinabove is defined by the appended claims and all changes to the disclosed concepts that fall within the meaning and range of equivalency of the claims are to be embraced within their scope.

Although the above systems, methods and various embodiments and aspects thereof have been described in detail for the purpose of illustration based on what is currently considered to be the most practical and preferred embodiments, it is to be understood that such detail is solely for that purpose and that the present disclosure is not limited to the described embodiments or aspects but, to the contrary, is intended to cover modifications and equivalent arrangements that are within the spirit and scope of the present system. For example, it is to be understood that the present disclosure contemplates that, to the extent possible, one or more features of any embodiment or aspect can be combined with one or more features of any other embodiment or aspect. In fact, any of these features can be combined in ways not specifically disclosed in the specification.

1. A system for use in delivery of fluid to a patient, the system comprising:
a first tubing set having a first length of tubing and a second length of tubing, the first length of tubing and the second length of tubing each having a proximal end and a distal end;
a first connector member positioned at the proximal end of the first length of tubing, the first connector member configured to connect to a flushing syringe; and
a valve assembly positioned at the distal end of the first length of tubing, the valve assembly comprising:
a proximal port, a distal port, and a third port;
a second connector member connecting the distal end of the first length of tubing to the proximal port;
a third connector member connected to the distal port; and
a fourth connector member connecting a first swabable valve and the second length of tubing to the third port,
wherein a container containing a medical fluid is removably connected to the first swabable valve, and
wherein the valve assembly selectively provides a fluid connection between the proximal port, the distal port, and the third port.

2. The system of claim 1, wherein the third connector member connected to the distal port is connected to a proximal end of a second tubing set,
wherein the second tubing set further comprises a one-way check valve configured to allow fluid flow toward the distal end of the second tubing set.

3-5. (canceled)

6. The system of claim 2, wherein the first connector member is connected to the flushing syringe and the flushing syringe is a manual syringe, an auto-injector, or a syringe connected to a fluid injection system.

7. The system of claim 6, wherein the flushing syringe contains a flushing fluid, and
wherein the valve assembly provides a fluid connection between the proximal port of the first tubing set and distal port of the second tubing set, and actuation of a plunger or a piston of the flushing syringe pushes the flushing fluid through the second tubing set to flush the administration line.

8. The system of claim 1, wherein at least one of the first connector member, the second connector member, the third connector member, and the fourth connector member comprises at least one of a one way check valve and a luer connector.

9. The system of claim 1, wherein the first length of tubing is sized to hold a volume of up to about 30 mL of the medical fluid.

10. (canceled)

11. The system of claim 9, wherein the first length of tubing is sized to a hold a volume ranging from 0.1 mL to about 5 mL of the medical fluid.

12. The system of claim 1, wherein the first length of tubing is comprised of a translucent polymeric material such that the medical fluid in the first length of tubing is visible to a user or a sensor of a fluid injection system.

13. The system of claim 1, wherein the first length of tubing further comprises indicia configured to provide a volumetric measurement of the amount of medical fluid in the first length of tubing.

14. The system of claim 1, further comprising a volume control element positioned between the container containing the medical fluid and the first length of tubing, and
wherein the valve assembly provides a fluid connection between the third port and the proximal port, and the volume control element selectively controls fluid flow between the container and the first length of tubing such that a measured volume of the medical fluid flows from the container into the first length of tubing.

15. The system of claim 1, wherein the container containing the medical fluid is a second syringe, and
wherein the valve assembly provides a fluid connection between the third port and the proximal port, and the second syringe selectively controls fluid flow between the second syringe and the first length of tubing such that a measured volume of the medical fluid flows from the second syringe to the first length of tubing.

16. The system of claim 1, wherein a spike is connected to the swabable valve of the third port,
wherein the container containing the medical fluid is pouch, a bag, a bottle, or another vessel configured to receive the spike, and
wherein when the container receives the spike it is fluidly connected to the valve assembly.

17. The system of claim 1, wherein the first connector member is connected to the flushing syringe connected to a fluid injection system, and the fluid injection system is programmed or controlled to actuate a piston of the flushing syringe to draw a predetermined volume of the medical fluid from the container into the first length of tubing.

18. The system of claim 15, wherein the second syringe comprises a pulsing syringe configured to selectively control fluid flow from the second syringe to the first length of tubing by delivering a predetermined volume of the medical fluid a predetermined number of times from the pulsing syringe into the first length of tubing.

19. (canceled)

20. The system of claim 18, wherein the predetermined volume of the medical fluid ranges from 0.1 mL to about 1 mL.

21. A system for use in delivery of fluid to a patient, the system comprising:
a first length of tubing having a proximal end and a distal end;
a first syringe connected to the proximal end of the first length of tubing;
a valve assembly positioned at the distal end of the first length of tubing, the valve assembly comprising:
a first port, a second port, and a third port;
connector members connected to each of the first port, the second port, and the third port; and
a first swabable valve connected to the third port;
wherein the second port is connected to a proximal end of a second tubing set and a catheter configured to be fluidly connectable to a patient,
wherein the distal end of the first length of tubing is connected to the first port,
wherein a container containing a medical fluid is removably connected to the valve assembly.

22. The system of claim 21, wherein the system further comprises:
a second syringe; and
an additional connector member between the third port and the second syringe,
wherein the second syringe is removably connected to the additional connector member and the third port, and
wherein the connector members and the additional connector member are configured to selectively control fluid flow between the container, the second syringe, and the first length of tubing, such that a measured volume of the medical fluid is allowed to flow from the container to the first length of tubing, or from the second syringe to the first length of tubing.

23. The system of claim 22, wherein the container is disconnected from the swabable valve and the swabable valve receives a valve cap, or wherein the second syringe is disconnected from additional connector member and the additional connector member receives a connector member cap.

24. The system of claim 21, wherein the system further comprises a third length of tubing having a proximal end and a distal end, and the container comprises a pre-filled component positioned between the first length of tubing and the valve assembly,
wherein the proximal end of the third length of tubing is connected to the first syringe, and the distal end of the third length of tubing is connected to the second tubing set,
wherein the third length of tubing is a bypass line configured to deliver a flushing fluid to the second tubing set and to the patient, and
wherein the flushing fluid is delivered through the first length of tubing and the pre-filled component is actuated to deliver the medical fluid through the first length of tubing to the second tubing set and to the patient.

25-32. (canceled)

33. A method of using a fluid injection system, the method comprising:
providing a fluid injection system comprising:
a housing, at least one syringe, at least one drive member, and at least one controller programmed or configured to actuate the at least one drive member;
an administration line;
a first length of tubing having a proximal end and a distal end;
a first connector member positioned at the proximal end of the first length of tubing, the first connector member configured to connect to the at least one syringe; and
a valve assembly positioned at the distal end of the first length of tubing, the valve assembly comprising:
a proximal port, a distal port, and a third port;
a second connector member connecting the distal end of the first length of tubing to the proximal port;
a third connector member connected to the distal port and configured to connect to a proximal end of the administration line; and
a fourth connector member connecting a first swabable valve to the third port;
priming the at least one syringe by pushing a flushing fluid into the at least one syringe;
connecting the administration line to the third connector member and the proximal port of the valve assembly;
connecting the proximal end of the first length of tubing to the at least one syringe of the fluid injection system;
priming the fluid injection system by actuating the at least one drive member and pushing a flushing fluid contained in the at least one syringe through the first length of tubing and through the administration line;
connecting a container containing a medical fluid to the first swabable valve;
actuating the at least one drive member to draw a volume of the medical fluid into the first length of tubing; and
actuating the at least one drive member to push the volume of medical fluid from the first length of tubing through the administration line and to the patient.
